(19) United States
(12) Patent Application Publication
(10) Pub. No.: US 2011/0160575 A1
(43) Pub. Date: Jun. 30, 2011

(54) MICROPOROUS BALLOON CATHETER

(75) Inventors: Mordechay Beyar, Caesarea (IL); Oren Globerman, Kfar-Shemaryahu (IL); Hila Wachsler-Avrahami, Tel-Aviv (IL)

(73) Assignee: By-pass, Inc., Orangeburg, NY (US)

(21) Appl. No.: 13/061,710

(22) PCT Filed: Sep. 2, 2009
(86) PCT No.: PCT/IL09/00850

§ 371 Date: Mar. 2, 2011

Related U.S. Application Data
(60) Provisional application No. 61/193,886, filed on Jan. 5, 2009.
(60) Provisional application No. 61/093,467, filed on Sep. 2, 2008.

(51) Int. Cl. A61M 25/10; A61B 6/00

(52) U.S. Cl. 600/424; 604/103.01; 604/98.01; 604/509

(57) ABSTRACT

A method and system for delivering a medicament into tissue, for example via a balloon with small pores. Optionally, the pressure used is high enough to cause jetting, but the pore sizes, pore density, pressure and/or delivery time are such that the jetting do not cause unacceptable tissue damage. Optionally, the pores are smaller than 2 microns, are between 300 and 600 per square centimeter and the pressure is above 8 atmospheres.

### RELATED PATENTS

This application is related to U.S. Provisional Application for Patent No. 61/093,467, filed on Sep. 2, 2008, and U.S. provisional application No. 61/193,886, Filed Jan. 5, 2009 the disclosures of which are incorporated herein by a reference.

### FIELD OF THE INVENTION

The present invention, in some embodiments thereof, relates to systems and methods for dispensing medicament or other materials into a body passage wall or cavity.

### BACKGROUND OF THE INVENTION

Balloon catheters are widely used for opening stenotic or occluded body passages, including blood vessels. Such balloons also serve as delivery apparatus for stents which mechanically keep the body lumen open.

Restenosis is a side effect that follows angioplasty treatments in blood vessels and also in other body lumen that is mechanically forced to expand (such as by Percutaneous Transluminal Coronary Angioplasty, PTCA). In order to prevent restenosis, local drug delivery of special medicament is sometimes performed during or after PTCA. Until recently, Heparin was widely used as such medicament due to its anticoagulant character, however in recent years efforts are more focused in the use of cells division reduction (anti-proliferative drugs), for example using Paclitaxel or Sirolimus. Drug-eluting stents, usually coated with anti-proliferative agent, are becoming a major trend in today's PTCA treatments.

A different balloon catheter design is the drug-eluting balloon, which is a dilatation balloon coated with a medicament such as Paclitaxel (usually in the amount of a few micrograms per a square millimeter of a balloon surface). Such devices and method of treatment are described in US patent application 2006/0020243 and in Scheller et al., “Treatment of coronary in-stent restenosis with a paclitaxel-coated balloon catheter”, N Engl J Med 2006; 335:2113-24, and in Scheller et al., “Paclitaxel balloon coating, a novel method for prevention and therapy of restenosis”, Circulation 2004; 110:810-814, the disclosures of which are fully incorporated herein by reference. A potential disadvantage of this technique is that a significant part of the drug coating can peel off the balloon during its insertion and manipulation until reaching the treatment site. This can reduce the delivery reliability with the total volume of effective drug delivered at the treatment site being impossible to accurately control. Furthermore, in order to provide a desired minimal dose of medicament to the treatment site, the balloon needs to be coated with significantly more drug, which drug and which coating are expensive. Another potential drawback is any toxic effect of drug released into the drug stream rather than the vessel wall.

Drug-dispersing balloon catheters were mooted, between the late 1980s to and the 1990s, although none of these devices were found to produce sustainable results, possibly due to a combination of ineffective drugs, ineffective methods of deliveries, and problematic delivery mechanism designs. Most designs include a balloon catheter with a single or multiple balloons having a plurality of pores, through which the drug is dispersed during or after the angioplasty phase. Exemplary designs are described in U.S. Pat. Nos. 4,994,033, 5,611,775, 5,087,244, 5,232,444, 5,098,381, 5,213,576, 5,318,531, 5,498,238, 5,049,132 and 5,569,198, the disclosures of which are fully incorporated herein by reference.

The Wolinsky perforated balloon catheter (of C.R. Bard, Inc.; also described in U.S. Pat. No. 5,087,244), with twenty-eight holes, each having a diameter of 25 μm, describes a macroporous balloon for delivery medicament into blood vessel walls. According to Lincoff et al. (“Local drug delivery for the prevention of restenosis”, Circulation, 90:4, October 1994; the disclosure of which is incorporated herein by reference), the inflation of this balloon causes streaming of infusate through the holes into the opposed vessel wall—throughout the arterial media and into adventitia, with the depth of delivery related to infusion pressure. According to Lincoff et al., this device has suffered from a potential for vascular trauma from the fluid jets and this may be related to the infusate pressure. Additionally, the pores are described as tending to become obstructed.

Other designs of macroporous balloons were later introduced, using a multiple-balloons formation (e.g., including an inner inflation balloon and an outer perforated balloon for drug administration), to allow angioplasty and drug infusion, using the same catheter (see for example U.S. Pat. No. 5,611,775). This design allows PCTA balloon inflation pressure to be disassociated from drug delivery—so that drug delivery could be performed with much lower pressures and velocities.

The following table summarizes the main claimed parameters apparently described in some publications for macroporous balloons:

| | | | | Max- | | |
|---|---|---|---|---|---|---|
| | | Hole | | imal | | |
| Patent | Balloon | Diam- | Holes | Pres- | | |
| Number | Design | eter | Density | sure | Velocity | Flow Rate |
| 5,087,244 | Single | 25 μm | | 2-5 | | 2-12 |
| | Chamber | | | Atm | | cc/min |
| 4,994,033 | Double | Laser | | 7-10 | | |
| | Balloon | drilled | | Atm | | |
| | | holes | | | | |
| 5,611,775 | Double | 10-100 | 15-100 | | 0.5-15 | 0.0015-0.05 |
| | Balloon | μm | holes/ | | m/sec | cc/sec per |
| | | | cm² | | | hole |

According to U.S. Pat. No. 5,569,198 (to Racchini), one cannot avoid causing trauma (“jetting effect”) without moving to a microporous balloon design that is intended for low-flow-low-speed drug delivery. The Racchini exemplary design provides a single chambered balloon catheter with a microporous membrane that contains 10⁸ pores, each having a 0.1 μm diameter. Apparently, by comparing the flow rate and fluid velocity obtained with his balloon design to those achieved by a macroporous balloon, at a given pressure, Racchini shows that changing to low-flow-low-speed drug delivery results in less leakage of fluids during the inflation stage and that the fluid exit velocity is so much lower, that the potential for trauma is reduced. Also, apparently, according to Racchini, the microporous balloon allows more uniform delivery. Other microporous infusion balloon designs are described in U.S. Pat. Nos. 5,213,576, 5,318,531 and 5,498,238. An additional microporous balloon design is the Atrium's Coronary ClearWay, in which an inflation balloon is covered with thin microporous PTFE balloon. During balloon inflation, drug is infused through the pores at low pressures (1-4 atmospheres).

The following table summarizes the main claimed parameters apparently described in some publications for small pore balloons:

| Pat. | Balloon | Hole | Holes | Maximal | |
|---|---|---|---|---|---|
| No. | Design | Diameter | Density | Pressure | Flow Rate |
| 5,498,238 | Single | ≦1 μm | Not | <6.2 Atm | Not mentioned |
| | Chamber | | mentioned | | |
| 5,569,198 | Single | 0.001-1 μm | >10,000 holes/cm² | 1-12 Atm | Flux Rate: |
| | Chamber | | | | 0.001-0.4 ml/(min · cm² · Atm) |

| | | | | |
|---|---|---|---|---|
| 5,318,531 | Macroporous | Membrane: | Membrane: | The membrane was evaluated using |
| | Balloon | 0.4-3 μm | 100,000-5,000,000 holes/cm² | water at a pressure of 10 psi. |
| | Covered | Balloon: | Balloon: | Obtained flow rate was about |
| | with a | 5-100 μm | 20-1,000 holes/cm² | 215 cc/min/cm² |
| | Microporous | | | |
| | Membrane | | | |

Several papers have been published regarding the mechanism of jetting of fluid, including:

Baxter et al. of “Jet injection into polyacrylamide gels: investigation of jet injection mechanics”, Journal of Biomechanics 37 (2004), 1181-1188; and in “Jet-induced skin puncture and its impact on needle-free jet injections: experimental studies and a predictive model”, Journal of Controlled Release 106 (2005), 361-373; and “Needle-free jet injections: dependence of jet penetration and dispersion in the skin on jet power”, Journal of Controlled Release, 97 (2004), 527-535; the disclosures of which are fully incorporated herein by reference.

Urethra stricture is a chronic disease which is being treated by widening, or by widening and incision of the stenotic area. In severe cases, reconstruction of the urethra (urethroplasty) is performed during a surgery. Nevertheless, re-narrowing of the urethra is often seen, and occasionally re-dilatation of the stricture is required. Several attempts have been made to inject drugs (such as Colchicine, Triamcinolone and anti-proliferative agents) for the prevention of urethra re-stricture. Using a syringe, the drug was injected percutaneously, transperineally, to the stricture site. In addition, tests performed in animals indicated that Paclitaxel eluted from drug-coated stents in the urethra have the potential to reduce tissue hyperplasia (Ji Hoon Shin et al., Tissue Hyperplasia: Influence of a Paclitaxel-eluting Covered Stent—Preliminary Study in a Canine Urethral Model, Radiology 2005 (234): 438-444).

### SUMMARY OF THE INVENTION

The present invention, in some embodiments thereof, relates to a method and system for delivering a medicament into tissue, for example via a balloon with small pores. Optionally, the pressure used is high enough to cause jetting, but the pore sizes, pore density, pressure and/or delivery time are such that the jetting do not cause unacceptable tissue damage. Optionally, the pores are smaller than 2 microns, are between 300 and 600 per square centimeter and the pressure is above 8 atmospheres.

A broad aspect of some embodiments of the invention relates to drug (or other medicament) delivery using an apertured delivery system, in which the aperture design and delivery pressure are selected to simultaneously reduce jetting damage, while providing penetration into tissue using jets. In an exemplary embodiment of the invention, jetting damage is reduced by using smaller apertures. Optionally or alternatively, sufficient depth is provided using a high enough pressure and thereby jet speed (possibly depending on aperture design), to achieve a desired penetration. Optionally or alternatively, the amount of material delivered is high enough to achieve a desired effect and generally higher than possible using small pores and low pressure, while possibly not as high as possible with large apertures and high pressure. In an exemplary embodiment of the invention, the manner of delivery is such that tissue trauma such as tissue damage, edema formation, vessel rupturing, and/or other damage, for example damage which can induce restenosis, is avoided or reduced, for example, by 20%, 50%, 80% or more or intermediate percentage (on the average) as compared to no treatment.

There is provided in accordance with an exemplary embodiment of the invention, a medicament delivery system comprising a delivery unit, comprising:

a chamber having at least one wall, wherein said wall defines at least 10 pores with a pore diameter between 1 to 5 μm and a surface pore density of 300-10,000 pores/cm².

In an exemplary embodiment of the invention, said chamber comprises a balloon. Optionally, said balloon is mounted on a catheter.

In an exemplary embodiment of the invention, the system comprises a pressure source fluidicly connected to said chamber.

In an exemplary embodiment of the invention, the system comprises a filter between said pressure source and said wall, said filter configured to pass particles smaller than 2 microns. Optionally, said pressure source is configured to provide both a pressure suitable for expanding a passageway and a delivery pressure of at least 4 atmospheres greater than said passageway expanding pressure. Optionally, said delivery pressure is sufficient to cause jetting of a medicament contained in said chamber, with properties suitable for penetrating into a blood vessel wall. Optionally, the system comprises a controller which controls one or both of said delivery pressure and a duration of said delivery to control a depth of penetration. Optionally, said duration is between 5 and 60 seconds.

In an exemplary embodiment of the invention, said jetting is suitable for penetrating past said blood vessel wall.

In an exemplary embodiment of the invention, the system contains an anti-proliferative agent suitable for the prevention of restenosis.

In an exemplary embodiment of the invention, the system is sized for insertion into one or more of a urethra, a trachea, a ureter, an eosophagus, an ileum, a biliary duct, a fallopian tube, a tear duct and a nasal cavity.

In an exemplary embodiment of the invention, said pore size is between 1.4 and 2 microns. Optionally or alternatively, said pore density is between 300 and 600 pores/cm² for an area of at least 0.5 cm². Optionally or alternatively, said pore density is about 550 pores/cm² and said pore size is about 1.7 microns in diameter. Optionally or alternatively, said chamber is pressurized to at least 15 atmospheres. Optionally or alternatively, said chamber is non-compliant.

In an exemplary embodiment of the invention, said chamber is filled with a fluid including a plurality of particles configured to slowly release a medicament.

In an exemplary embodiment of the invention, the system is packaged as a kit with medicament suitable for treating tissue.

In an exemplary embodiment of the invention, at least 70% of said pores are oriented within 20 degrees of a perpendicular to said membrane.

In an exemplary embodiment of the invention, the system contains a radio-opaque material in an amount suitable for fluoroscopic imaging, adjacent or in said chamber.

In an exemplary embodiment of the invention, the system comprises a unit which displays an estimate of an actually delivered amount of medicament.

In an exemplary embodiment of the invention, said wall defines at least 100 pores and wherein at least 90% of pores in said wall are smaller than 5 microns in diameter.

There is provided in accordance with an exemplary embodiment of the invention, a medicament delivery system comprising a balloon having a plurality of at least 50 pores formed therein, said pores having a diameter of less than 5 microns, said balloon being filled with a medicament under a pressure suitable for causing jetting of said medicament through said pores into tissue to a depth of at least 0.1 mm. Optionally, said pores have a diameter of less than 2 microns. Optionally or alternatively, said pressure is at least 8 atmospheres.

In an exemplary embodiment of the invention, said balloon is suitable for PCTA. Optionally or alternatively, said medicament is suitable for preventing restenosis when injected into vascular tissue. Optionally or alternatively, said medicament includes radio-opaque contrast medium.

There is provided in accordance with an exemplary embodiment of the invention, a method for treating a narrowed segment of a body lumen, comprising:

(a) locating an inflatable member having a plurality of pores smaller than 10 microns formed in a surface thereof, adjacent said segment;

(b) inflating said member with a pressure of an amount sufficient to widen said narrowing and not sufficient to cause jetting out of said pores; and

(c) further inflating said member with a higher pressure which does not significantly increase a diameter of said member but is sufficient to cause jetting of medicament out of said pores and into said tissue to a depth of at least 50 microns.

Optionally, said surface has a pore density in the range of 300-10,000 pores/cm² for an area of at least 0.5 cm² and pore diameters in the range of 1-5 μm. Optionally or alternatively, the method comprises applying said jetting at a velocity and for a time suitable to form medicament reservoirs in said tissue. Optionally or alternatively, said medicament is configured to adhere to said narrowing. Optionally or alternatively, said inflating and said further inflating are part of a continuous inflation act.

In an exemplary embodiment of the invention, the inflation pressure is between 5 and 12 atmospheres. Optionally or alternatively, the further inflation pressure is between 10 and 50 atmospheres.

In an exemplary embodiment of the invention, said further inflation lasts between 5 and 60 seconds.

In an exemplary embodiment of the invention, the medicament includes an anti-proliferative agent in an amount suitable for the prevention of restenosis.

In an exemplary embodiment of the invention, an amount of leaking medicament during said inflating is less than 20% of an amount existing said member by said further inflating.

In an exemplary embodiment of the invention, the method comprises deploying a stent in said narrowing using said inflatable member.

In an exemplary embodiment of the invention, the method is for the treatment or prevention of in-stent restenosis.

In an exemplary embodiment of the invention, the method is for the treatment of a blood vessel following arterectomy.

In an exemplary embodiment of the invention, the method comprises displaying at least an estimation of jetted medicament to a user during said further inflation, in real time.

In an exemplary embodiment of the invention, the method comprises imaging said narrowing during said further inflation, using a radio-opaque material adjacent or coupled to said inflatable member.

In an exemplary embodiment of the invention, said further inflating does not cause tissue damage significant enough to cause restenosis.

In an exemplary embodiment of the invention, said further inflating comprises injecting at least 0.025 ml/cm² medicament into tissue adjacent said narrowing.

In an exemplary embodiment of the invention, said further inflating comprises injecting at least 0.07 ml/cm² medicament into tissue adjacent said narrowing.

In an exemplary embodiment of the invention, said pores have a diameter less than 5 microns.

In an exemplary embodiment of the invention, said medicament slowly releases into tissue, over a period of at least 5 days.

There is provided in accordance with an exemplary embodiment of the invention, a method for treating a body portion, comprising:

(a) locating a chamber having a plurality of pores smaller than 5 microns formed in a surface thereof, adjacent said portion;

(c) pressurizing said chamber with a pressure which is sufficient to cause jetting out of said pores and into said tissue to a depth of at least 50 microns.

There is provided in accordance with an exemplary embodiment of the invention, a method for producing a microporous balloon having at least 50% of pores at a desired orientation, comprising:
providing at least one perforation source;
shielding at least a part of a membrane surface that is not oriented within a desired angular range of said at least one perforation source;
activating said at least one perforation source;
exposing a different portion of said membrane to said at least one perforation source; and
   repeating said activating and said exposing until a desired pattern of perforations or nascent perforations are formed in said membrane. Optionally, said membrane is a balloon and wherein said exposing comprises rotating said balloon. Optionally or alternatively, said desired orientation and said at least one perforation source are selected so that said perforation or nascent perforations are substantially perpendicular to a surface of said membrane. Optionally or alternatively, said source comprises a radiation source suitable for weakening a molecular structure of said membrane and comprising chemical etching of said membrane to convert nascent perforations formed by said weakening into perforations. Optionally or alternatively, said perforations or nascent perforations have a diameter of less than 5 microns and said membrane has a thickness of at least 10 microns.

There is provided in accordance with an exemplary embodiment of the invention, a method for drug delivery using microporous balloon catheter, comprising:

increasing a pressure of a fluid to at least a pressure suitable for delivery;

filtering said fluid; and

extruding said filtered fluid through pores in a microporous balloon. Optionally, extruding comprises extruding as jets which penetrate into tissue and form fluid reservoirs therein. Optionally or alternatively, filtering comprises filtering after said fluid passes a perimeter of the human body.

There is provided in accordance with an exemplary embodiment of the invention, a drug delivery system, comprising:

a delivery head including a chamber with at least 50 pores each having a diameter of less than 5 microns formed therein;

a pressure source capable of reaching over 4 atmospheres and fluidicly connected to said chamber; and

a filter fluidicly located between said pressure source and said delivery head and configured to pass only particles smaller than 2 microns. Optionally, said filter is adjacent or in said delivery head. Optionally or alternatively, said head comprises an intravascular catheter.

There is provided in accordance with an exemplary embodiment of the invention, a pressurized medicament delivery perforated balloon that is filled with medicament and a fluidic contrast medium, in relative amounts that produces both radiographic visibility of said balloon when within a body and a viscosity suitable for jetting into tissue via pores of said balloon at a pressure under which said balloon is pressurized. Optionally, said mixture includes an anti-proliferative agent used for the prevention of restenosis.

There is provided in accordance with an exemplary embodiment of the invention, a method of selecting a mixture for a pore-based medicament delivery system, comprising:

selecting a medicament delivery perforated balloon and a desired treatment;

determining a desired viscosity for said treatment; and

formulating a mixture having said viscosity by mixing at least a medicament and a contrast material. Optionally, said formulating comprises also mixing a diluting material. Optionally or alternatively, said formulating and said determining are according to one or more of the balloon's average pores diameter, balloon number of pores, medicament delivery pressure and medicament delivery duration.

There is provided in accordance with an exemplary embodiment of the invention, an angioplasty and drug delivery balloon catheter comprising a plurality of pores with a pore diameter between, for example, 0.1 microns and 10 microns, for example, 1 to 5 μm and pores density of, for example, 300-10,000 pores/cm².

In an exemplary embodiment of the invention, the balloon is capable of being inflated under a standard angioplasty pressure, and of being further pressurized to a pressure higher in at least 4 atmospheres than said angioplasty pressure, and where most of the delivered drug, from the interior of said balloon through its pores, is being delivered under said higher pressure. Optionally or alternatively, at least part of the drug delivered from the interior of said balloon through its pores pierces the blood vessel wall tissue. Optionally, the drug pierces the vessel wall tissue and penetrates into the intima layer. Optionally or alternatively, the drug pierces the vessel wall tissue and penetrates into the media layer. Optionally or alternatively, the drug pierces the vessel wall tissue and penetrates into the adventitia and even beyond.

In an exemplary embodiment of the invention, the catheter is capable of performing active diffusion of a fluid from the interior of said balloon into a blood vessel wall by producing continuous streams of said fluid through its pores.

In an exemplary embodiment of the invention, said drug includes at least Paclitaxel, or other anti-proliferative agent used for the prevention of restenosis, such as Sirolimus (or its derivatives).

In an exemplary embodiment of the invention, the catheter is configured for dilatation of body passage strictureand/or prevention of restenosis, where said body passage may include a blood vessel or other passages, such as the urethra, trachea, ureter, prostate, eosophagus, ileum, biliary duct, ovaries, tear duct and nasal cavity. Optionally or alternatively, the catheter is adapted for the treatment of body organ in which administration of medicament to a localized area is required.

In an exemplary embodiment of the invention, at least substantial portion of said balloon pores is substantially perpendicular to balloon wall. Optionally or alternatively, a radiopaque material is incorporated into—and/or placed over balloon membrane to provide membrane visualization under imaging device operation.

In an exemplary embodiment of the invention, at least a portion of the drug is delivered coupled to a carrier that enhances its delivery and/or solubility.

In an exemplary embodiment of the invention, at least a portion of the drug is delivered encapsulated to provide for its slow release following administration.

In an exemplary embodiment of the invention, the balloon catheter further comprises a pressure gauge, a time measuring device and/or a unit capable of integrating injection parameters, where said integration is being translated to the amount of delivered drug, and where said delivered drug amount is displayed to the user.

In an exemplary embodiment of the invention, said catheter comprises a filter through which the drug solution passes prior to entering the balloon, and where said filter is capable of withstanding high pressure generated within the balloon.

There is provided in accordance with an exemplary embodiment of the invention, a drug delivery perforated balloon that is filled with medicament and a fluidic contrast medium mixed with solution such as saline, in a specific ratio that produces both imagery capabilities of said balloon when within body and a desired mixture viscosity. Optionally, the desired viscosity of medicament solution is achieved by the addition of material other than contrast medium. Optionally or alternatively, said mixture viscosity is formulated according to the balloon's average pores diameter, number of pores, pressure and drug delivery duration. Optionally or alternatively, said mixture includes Paclitaxel agent or other anti-proliferative agent used for the prevention of restenosis, such as such as Sirolimus or its derivative.

There is also provided in accordance with an exemplary embodiment of the invention, a method for delivering medicament into, for example, body passage wall. In an exemplary embodiment of the invention, a method for widening a narrowed segment of a body lumen (e.g., blood vessel) and delivering medicament into said body lumen wall, comprises:

(a) providing a catheter with:

a catheter shaft having proximal and distal ends and at least one inner lumen extending therein;

an inflatable member on a distal portion of the catheter shaft that includes an interior, which is in direct communication with the catheter shaft lumen, wherein said inflatable member includes a microporous wall with holes density in the range of 300-10,000 holes/cm² and holes diameters in the range of 0.1-10 μm; and means to pressurize medicament through the shaft lumen to the interior of the inflatable member;

b) advancing said catheter through a body lumen of a patient until the inflatable member is positioned at a site therein having a narrowed segment;

c) pressurizing medicament into the interior of the inflatable member until a first inflation pressure is met so that the inflatable member inflates and the microporous wall is in direct contact with the body lumen wall;

d) further elevating the pressure of the inflatable member interior until a second, dilatation pressure is met so that the inflatable member widens the narrowed body lumen segment; and

e) further elevating the pressure of the inflatable member interior until a third, injection pressure is met so that most medicament is ejected through the microporous wall of the inflatable member in the form of a corresponding plurality of individual streams, each individual stream maintains a relatively constant velocity for a period of time that is sufficient to produce a hole in the body lumen wall and/or to enhance the diffusion of the medicament in proximity of the jet, where such pressure is higher than the stricture dilatation pressure.

Optionally, c and d are combined as a single act that includes a continuous pressurization until a preferred widening of the narrowed passage segment occurs. Optionally, acts c, d and e are combined as a single act that includes a continuous pressurization until a preferred injection pressure is set, wherein said injection pressure is substantially higher than the minimal pressure needed for a requested passage widening.

Optionally, the first inflation pressure is equal to—or smaller than 10 atmospheres, optionally smaller than 5 atmospheres. Optionally, the dilatation pressure is a recommended PTCA or other angioplasty pressure, optionally between 5 to 12 atmospheres. Optionally, the maximal injection pressure is between 10 to 50 atmospheres, optionally 15-30 atmospheres.

Optionally, acts c, d and e together last less than 90, optionally less than 60 seconds. Optionally act e lasts between 5 to 60 seconds, optionally between 10 to 30 seconds.

Optionally, during d (dilatation) a non-significant amount of medicament emerges through the balloon pores.

In an exemplary embodiment of the invention, the method comprises delivery of a stent incorporated onto said catheter, followed by stent opening in said narrowed segment of a body lumen.

In an exemplary embodiment of the invention, the method is intended for the treatment of in-stent restenosis and/or for the treatment of a blood vessel following arterectomy.

In an exemplary embodiment of the invention, the injected medicament amount is displayed to the user at real time and can be controlled by the user by adjusting the injection pressure and time.

There is provided in accordance with an exemplary embodiment of the invention, a method for producing a microporous balloon having a substantial portion of microholes angled at approximately 90 degrees to balloon wall, comprising:
shielding areas of balloon surface that are not substantially perpendicular to the perforation source;
activation of the perforation source so that only balloon portion which is approximately perpendicular to perforation source is exposed to said activation and is perforated;
rotating the balloon to locate previously-shielded balloon portion in a substantially perpendicular position relative to the perforation source while shielding other balloon portions which are not perpendicular to the perforation source, and re-activating the perforation source; and
repeating the last stage until a sufficient balloon surface area has been perforated.

There is provided in accordance with an exemplary embodiment of the invention, a filter capable of withstanding high pressure of up to 30 atmospheres, intended to purify a solution that is injected into the body.

There is provided in accordance with an exemplary embodiment of the invention, a method for drug delivery using microporous balloon catheter, comprising purifying the delivered medicament solution by passing said solution via a filter which is capable of withstanding high pressure and is connected to the delivery system.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow diagram illustrating an exemplary method according to some embodiments of the invention;

FIG. 1B is a schematic showing of a catheter treatment system in accordance with an exemplary embodiment of the invention;

FIGS. 2A-2F are schematic diagrams illustrating an operational sequence of a microporous balloon catheter according to some exemplary embodiments of the invention;

FIG. 3 is a schematic Pressure vs. Time graph illustrating an operational sequence of a microporous balloon catheter according to some exemplary embodiments of the invention;

FIG. 4 is a schematic illustration of a method to produce an exemplary perforated balloon according to an embodiment of the invention; and

FIG. 5 is a graph of fluid elution rate as a function of balloon pressure in accordance with an exemplary embodiment of the invention.

### DETAILED DESCRIPTION OF EXEMPLARY EMBODIMENTS

### Overview

A broad aspect of some embodiments of the present invention relates to a drug delivery balloon catheter capable of dispersing medicament or other materials as a plurality of minute jets while maintaining an overall useful flow rate under high pressures. In an embodiment of the invention, at least one jet produces a continuous impact with magnitude and duration that are high enough to perform an erosion hole in a body passage wall, optionally a blood vessel wall, of a patient. Alternatively, no holes are performed, but the high pressure provides an active diffusion of the medicament into the blood vessel wall.

In accordance with exemplary embodiments of the invention, there are provided methods and apparatus for delivering medicament to a localized area of a body passages (e.g., into blood vessel wall), for example, by producing erosion holes that are deep enough to reach at the arterial intima layer and at least the endothelium, and/or to reach at and/or in the arterial media, and optionally not penetrate through all said layers into the adventitia. Alternatively, the holes are deep enough and/or the jetting otherwise suitable to reach further, for example, into adventitia or beyond, for example to nearby tumors.

In an exemplary embodiment of the invention, the parameters of the method are selected and/or controlled so that, on the one hand, jet velocity (e.g., based on pressure, device design) is high enough to provide a desired penetration, while it is not so high and/or applied for so long as to cause over penetration. In an exemplary embodiment of the invention, the size (e.g., diameter) of the jet is controlled so that less damage is caused to the tissue, while the spatial density (e.g., percentage of tissue volume that is actually a fluid reservoir or otherwise medicament) and/or total amount of delivered medicament is controlled to be a useful amount of medicament.

Some embodiments of the invention are in contrast with prior art macroporous balloons where the jet is too large and causes too much tissue damage and also in contrast to prior art microporous balloons.

Some embodiments of the invention are based on a discovery by the inventors that pressures higher than these claimed in U.S. Pat. No. 5,569,198 may be beneficial in delivery significantly higher quantities of medicament into tissue while still not causing substantial tissue damage (“jetting effect”). In some embodiments, hole (pore) size and/or density is changed as well, for example, using larger holes and/or lower density of holes

A potential advantage of some embodiments of the invention is that a lower pressure pressure-source may be used, or at least that as the medicament flow rate may be lower than in balloons with large pores, it is easier to maintain pressure in the balloon as the pressure drop caused by material existing is lower. Another potential advantage is that a single chamber balloon may be used, which balloon can be, for example, simpler to manufacture, easier to compress after the method and/or more flexible.

In an exemplary embodiment of the invention, the balloon catheter comprises a filter, which is intended to prevent blockage of balloon micropores by impurities in the delivered solution and which is capable of withstanding high pressure. Optionally, said filter is placed at a proximal section of the catheter, or at a distal end, optionally, within the balloon or at an interface between the balloon and a shaft of the catheter. In other embodiments, the filter is provided outside of the catheter. In an exemplary embodiment of the invention, the filter comprises a perforated film, enclosed within a housing. In an exemplary embodiment of the invention, the pores diameter of the filter may be in the range of 0.1-3 μm, optionally in the range of 0.5-1.5 μm. Optionally, the filter passes particles that are less than 80%, 60%, 30%, 10% or smaller or intermediate percentages of the pore sizes. In an exemplary embodiment of the invention, the filter passing size is between 0.2 and 0.8 microns, for example, about 0.45 microns. In an exemplary embodiment of the invention, the filter area size is between 0.1 cm² and 4 cm², for example, 2 cm².

Optionally, a non-sieve filtering mechanism is used, for example, a centrifugal filter or a sorting filter where particles that are too large are washed away from apertures sized to pass correctly sized particles.

In an exemplary embodiment, the filter film and the filter housing are made of material to which the drug does not adhere or otherwise interact with. Optionally, the filter film and the filter housing are made of Polycarbonate or other material capable of withstanding high pressure. In an exemplary embodiment of the invention, the filter is an elongate filter, for example, between 2 and 20 mm long with a diameter that is equal to or less than the length, for example, less than 50%, or 30% of the length. This may be useful if the filter diameter is small, for example, if the filter fits in the catheter or the balloon. Optionally, the use of a filter prevents pores form being blocked. Which prevention may assist in providing a more uniform and/or otherwise desired delivery of medicament to tissue.

In an exemplary embodiment of the invention, balloon visualization during the procedure is enabled by using a radioopaque material which is provided with the balloon. For example, at least one thread, bar, strip, ring, dot or other form of radioopaque material, such as Tantalum, is provided within and/or over the balloon surface or adjacent the balloon. Optionally, this allows reducing the use or avoiding the use of contrast medium. Optionally, multiple radioopaque markers are provided or a single elongate marker is positioned so that the expansion of the balloon can be measured under fluoroscopy/X-rays.

In an exemplary embodiment of the invention, a catheter balloon treatment system is configured to provide a user with the information of the amount of medicament delivered and/or other delivery parameters, such as delivery pressure and time.

In an exemplary embodiment of the invention, a physician can choose and/or control of the desired amount of drug to be delivered, for example, by adjusting the parameter(s) of pressure and/or time for each drug injection. This may be important in cases where, for example, a patient requires a higher dose of medicament. In an exemplary embodiment of the invention, the system includes a unit (e.g., calculator or printed table) that interrelates various injection parameters and the amount of medicament delivered. In an exemplary embodiment of the invention, a digital pressure gauge and a time measuring device are provided. Integration of the pressure and time parameters can give real time medicament flow rate, which can be translated to the delivered amount of medicament, optionally after correcting for elasticity effects in the delivery catheter and balloon. Optionally, such a unit serves to calculate and/or display desired treatment and/or actual treatment. Optionally, such a unit displays a total amount delivered, a sequence of pressure pulses to be applied and/or a sequence of such pulses that was actually applied.

In an exemplary embodiment of the invention, the drug delivery catheter serves for delivering an implant, for example, a balloon-mounted stent to be placed and opened in a narrowed region. In an exemplary embodiment of the invention, the medicament delivery process further includes the opening of the stent from a collapsed to a widened form before or after medicament delivery. Optionally, stent design (e.g., stent's struts design) and/or balloon design (e.g., perforation pattern and/or hole design) incorporate an improved correlativity and efficiency, for example, avoiding blocking more than, for example, 10%, 20%, 30% or intermediate percentages of pores in the balloon by the stent. It should be noted that medicament delivery can be via a device other than a balloon, for example, via one or more non-expanding porous tubes, a flat, optionally curved surface (e.g., rigid or flexible, optionally comprising a chamber covered by a membrane at least in part. Optionally, such a delivery system is thin, for example, having a thickness (e.g., minimal dimension and/or minimal trans-axial dimension) of less than 5 mm, 2 mm, less than 1 mm and/or less than 0.5 mm or intermediate sizes.

In another embodiment of the invention, the suggested balloon catheter may be used to inject a medicament following arterectomy or other treatment of a blood vessel. Optionally, a same balloon is used for vascular tissue ablation and for material delivery, for example, including both cutting wires or ridges and pores.

While in an exemplary embodiment of the invention the body part treated is a blood vessel, a medicament delivery system as described herein can be used for other body parts as well, for example, for treating a body passage or tissue adjacent such a passage or adjacent an artificially created cavity. Example tissues include any live tissue, tumor or organ that is adjacent to a body passage, for example into a prostate or into a heart. Exemplary body passages which may be treated and/or through which treatment may be delivered to adjacent tissue include, for example, blood vessels (e.g., coronary or peripheral, veins or arteries), urethra, trachea, ureter, prostate, eosophagus, ileum, biliary duct, ovaries, tear duct, and/or a nasal cavity. Optionally or alternatively, the passageway is artificial, for example, formed by a separate instrument and/or by the drug delivery device itself (e.g., by its being pushed into tissue to form a passageway, optionally the device including a cutting tip). In an exemplary embodiment of the invention, the catheter delivery system is adapted for the passageway and/or tissue treatment. For example, the rigidity, length or diameter of the catheter may be changed. Optionally or alternatively, the length and/or diameter of the balloon may be changed. Optionally or alternatively, the number and/or size and/or positioning and/or density of the pores may be changed. Optionally or alternatively, the pressure protocol used may be changed. Optionally or alternatively, duration and/or viscosity and/or active ingredient concentration, may be changed. Optionally or alternatively, any of these parameters may be changed to take into account the medicament being delivered.

In an exemplary embodiment of the invention, the delivery system includes a membrane material. Optionally, the membrane is perforated using a track-etching technique, although other perforation methods known in the art may be used. Optionally, the balloon is made, for example, from Nylon, and is perforated using a laser.

In an exemplary embodiment of the invention, membrane perforation is made so that at least a substantial portion (e.g., 50%, 60%, 70% or more) of the perforations is perpendicular (e.g., within 30 degrees, 20 degrees, 10 degrees, and/or 5 degrees of a perpendicular) to the balloon wall at the point of perforation. This may provide more efficient delivery of medicament into tissue. Optionally or alternatively, other controlled directions can be provided, for example, a plurality of pores (e.g., more than 3, more than 4 or more than 10) selected to have jets thereof meet at a point inside the tissue (focused arrangement), or diverging away form each other. For example, at least 3, 4, 5, or more focused or diverging pore arrangements may be provided. Optionally, such directionality is provided by using a source having diverging or converging beams, for example, using a suitably shaped source and a suitable aiming mask. In an exemplary embodiment of the invention, most of the surface area of the membrane (e.g., balloon) are shielded from a perforation source (e.g., a cyclotron), while a small portion of the balloon, having a substantial part that is positioned perpendicular to said perforation source, is exposed to the perforation source. Optionally, the balloon is rotated at a controlled rate (e.g., in steps), so that each portion of the rotated balloon is perforated separately, while most of it is approximately perpendicular to the perforation source. Optionally, the perforation is by ion beam used to weaken the membrane and then dipping in a chemical etchant, such as an acid, to remove weakened regions.

Also provided in accordance with some embodiments of the invention is the use of medicament mixtures for delivery via a small pore balloon, at a pressure sufficient to cause jets. Optionally or alternatively, such medicament includes contrast material and/or includes a desired viscosity. Optionally or alternatively, the medicament (or a kit including such medicament) is packaged with usage instructions and/or with a list of properties and/or mixing instructions relating, for example, to viscosity, treatment type and/or suitable delivery system.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### General System Overview

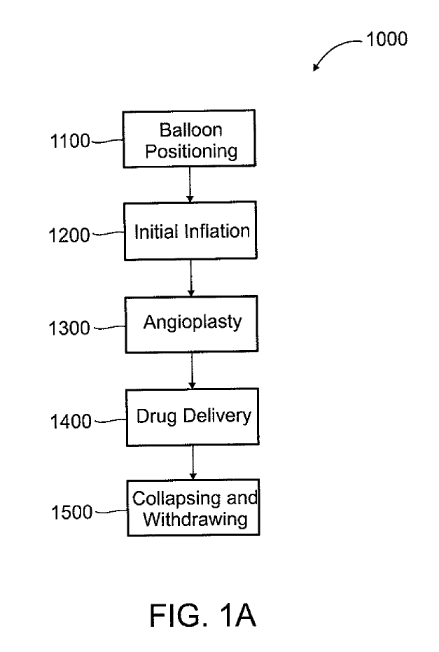

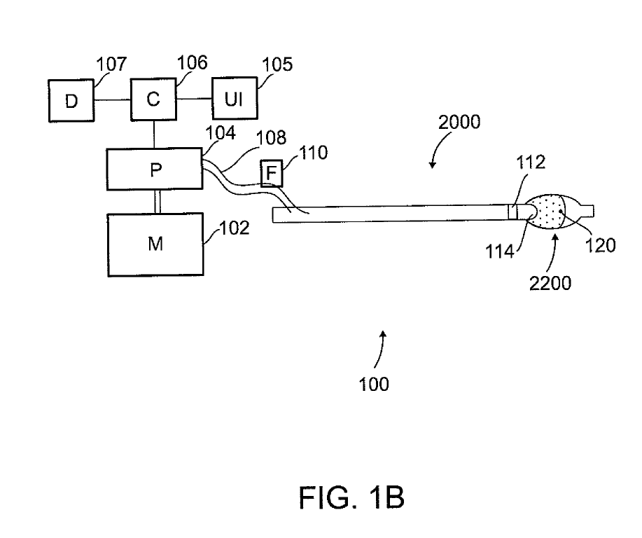

FIG. 1B is a schematic diagram of a catheter treatment system 100, in accordance with an exemplary embodiment of the invention.

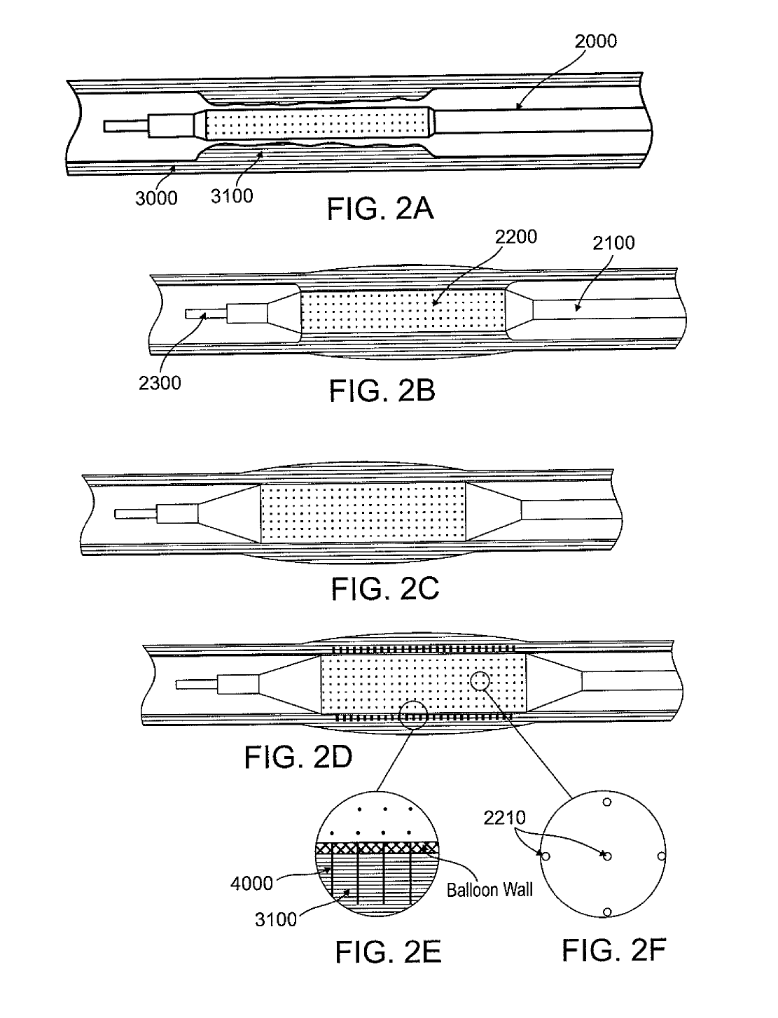

In an exemplary embodiment of the invention, system 100 includes a catheter 2000 (see FIG. 2), having a balloon expanding head 2200. Head 2200 can also be non-expanding or expanding on only at one portion thereof (e.g., an axial portion or a sector). Also, for example as described below, catheter 2000 may also be used to deliver a stent or provide other treatment to a vessel. While in an exemplary embodiment of the invention, catheter 2000 is flexible, for example, for use in coronary vessels, optionally, the shaft of catheter 2000 is replaced by or is connected to a rigid or semi-rigid shaft and/or handle.

In an exemplary embodiment of the invention, head 2200 includes one or more radio-opaque marker 120, for example, mounted on the shaft, mounted on the balloon membrane or inside the balloon. Optionally, a plurality of markers 120 (or an elongate marker) are positioned at either end of the porous areas of the balloon.

In an exemplary embodiment of the invention, a medicament is provided by a medicament source 102. Optionally, the source is incorporated inside a pressure source 104, but it may be separate and, for example pumped, from source 102.

An optional controller 106 may be used to control the pressure profile provided by the pressure source. Optionally, a display 107 (e.g., visual and/or auditory) and/or a user interface 105 (e.g., a keyboard, mouse and/or touch screen) are used, for example, to receive user settings and/or report on a progress to a user. Optionally or alternatively, controller 106 is a stand alone calculator which provides instructions to a user who then sets up system 100 accordingly and/or which provides a calculation of delivered dosages thereto.

An optional tubing 108 delivers the medicament under pressure to catheter 2000. In an exemplary embodiment of the invention, a filter (described below) is provided. Exemplary shown filters, not all of which or even one of which need be provided, are a filter 110 on tube 108, a filter 112 inside catheter 2000, optionally at a distal end thereof and a filter 114 within balloon 2200.

In exemplary use, which will be elaborated below, pressure source 104 delivers medicament from medicament source 102 under pressure via tubing 108 to balloon 2200 where it exits and optionally jets into adjacent tissue. Exemplary pressure forming means include a peristaltic pump, a syringe pump, a shockwave source an electric pump and/or a hydraulic pump.

In an exemplary embodiment of the invention, some or all of the delivery system is disposable and/or may be provided as a kit. For example, the balloon, the catheter, and/or pressure source pump may be disposable. Optionally, the computing unit, display and/or UI are reusable. Optionally, a pump does not contact medicament, except through a tube or other interface so it can be reused.

### Exemplary Balloon Catheter

FIGS. 2A-2B present schematic illustrations of a balloon catheter 2000 in accordance with some exemplary embodiments of the invention. In an exemplary embodiment of the invention, balloon catheter 2000 is positioned adjacent to narrowed segment 3100 of body passage 3000 (or other tissue to be treated). In an exemplary embodiment of the invention, balloon catheter 2000 includes a catheter shaft 2100 with an inner lumen (not shown) that is distally connected to a perforated balloon 2200. Optionally, a guide-wire 2300 may be used to assist with navigating to a treatment area and/or with balloon advancement within body, and may be coupled with balloon catheter 2000, for example, using an optional second inner lumen (not shown).

In an exemplary embodiment of the invention, balloon 2200 is made of a microporous membrane having pore density of 300-10,000 holes/cm², optionally 800-2,000 holes/cm². In an exemplary embodiment of the invention, 550 holes/cm² are provided. Exemplary balloon 2200 includes a substantially constant pore density throughout most of the balloon surface, although different exemplary balloons may be designed with several different or changing pore densities and/or may include specific surface areas that are not perforated. For example, a higher density may be provided on one or more angular sectors and/or axial sections. Optionally or alternatively, the arrangement of pores and/or other properties thereof may match a target to be treated. For example, pores may be concentrated (and/or be larger or smaller) at parts of the balloon at locations where greater stent pressure and/or restenosis are expected. In an exemplary embodiment of the invention, the pores are provided on parts of the balloon that are expected to contact a blood vessel wall (or other tissue), e.g., on parts parallel to the balloon axis. In some cases, the treated area is smaller than the stent area. Optionally, the balloon is replaced even after PTCA according to a desired treatment area.

In an exemplary embodiment of the invention, the average and/or minimal and/or maximal diameter of balloon pores 2210 (see also FIG. 2F, which is a schematic magnification of a portion of balloon 2200) is 0.1-10 μm, optionally 1-5 μm, optionally 1-3 microns. In an exemplary embodiment of the invention, the pores are of about 1.7 microns in diameter, or less or more, for example, 1-1.7 microns, 0.1-1 microns or 1.5-5 microns. Optionally, at least 80% of pores 2210 are substantially equal with a maximal allowed tolerance of 0.5 μm or less. Optionally, balloon 2200 includes different areas of different pore sizes (not shown). In an exemplary embodiment of the invention, different jet properties and/or pore density are provided at different parts of the balloon. Optionally, the balloon itself is asymmetric and/or non-cylindrical.

In an exemplary embodiment of the invention, smaller diameter holes provide a higher pressure threshold above which jetting occurs. Optionally, a plurality of different size holes (e.g., having a continuous size range or selected to have sizes from a set of discrete sizes) are provided so as to provide different jetting behavior at different pressures. Optionally, the pressures are selected according to pressure levels that a blood vessel can handle, desired penetration and/or PTCA pressures. Optionally, the balloon parameters are changed or selected according to tissue limitations. Optionally or alternatively, the pressures are selected according to the degree of non-compliance of the balloon. Optionally, the balloon expands less than 30% more, less than 20% more or less than 10% or 5% more in diameter when the pressure is doubled.

In an exemplary embodiment of the invention, balloon 2200 is a single chamber balloon that can be filled with fluid through catheter lumen 2100 by pressurizing means 104 (e.g., a manual PTCA pump, or other pump capable of providing higher pressures than a PTCA pump), that may be located outside patient body. Optionally, balloon 2200 is non-compliant (e.g., has a high modulus of elasticity) so that it does not substantially expand, but rather unfolds, when filled with fluid under pressure. Alternatively, in an exemplary embodiment of the invention, balloon 2200 is made of a relatively elastic material and/or designed as an expandable collapsed chamber, that is capable of expanding under relatively low pressures provided therein (e.g., 1 to 3 atmospheres), at least until its outer surface is in direct contact with body passage 3000 wall. Optionally, further expansion is prevented, for example, by a non-expanding mesh embedded in the balloon and/or covering it. Optionally, balloon 2200 is made of a biocompatible polymer such as Polyethylene terephthalate (PET).

Optionally or alternatively, pressure creating means such as described in U.S. application Ser. No. 11/335,317, published as 2006-0190022-A1, the disclosure of which is fully incorporated herein by reference are used to generate a pressure pulse.

Balloon 2200 porous material can be manufactured in any of several ways, most of which are readily understood by those skilled in the art of manufacturing microfiltration and ultrafiltration membranes. In an exemplary embodiment of the invention, the balloon material is perforated using a track-etch process, whereby a polymer film is bombarded by protons, ions, electrons or other radiation and then subsequently subjected to a controlled etching. Other exemplary manufacturing techniques are described in U.S. Pat. No. 5,498,238, the disclosure of which is fully incorporated herein by reference. Additional exemplary manufacturing methods and/or details are described below.

In order to withstand high pressures, specially designed balloons may be used. Optionally, the balloon wall width is over 15 μm, optionally over 20 μm, optionally over 50 μm, or of any intermediate value. Optionally, the balloon is reinforced, optionally by braiding or comprising a net embedded in the balloon material. Optionally, such a net prevents over-tearing of any holes/pores that tear during balloon inflation. The balloon may be produced from any biocompatible material, stretchable or non-stretchable.

Optionally, the balloon is made of polyethylene (PET), polycarbonate (PC), polyimide (PI), or other material which may be perforated with holes diameter and density as described in this application, and which is mechanically and biologically suitable for the discussed uses. Optionally or alternatively, other materials and/or manufacturing methods may be used to provided for a combination of material properties resulting in a balloon capable of withstanding 15 atmospheres, optionally 30 atmospheres, optionally 50 atmospheres, or higher or lower or any intermediate pressure.

In an exemplary embodiment of the invention, the balloon catheter includes an inflatable microporous member, with a specific fluid permeability chosen according to the viscosity of the fluid to be dispersed through the membrane and/or according to working pressure and/or according to desired jetting behavior. In an exemplary embodiment of the invention, the membrane holes density is less than 10,000 holes/cm², optionally in the range of 500-5,000 holes/cm², optionally 1,000-2,000 holes/cm², optionally 300-500 holes/cm², optionally 500-600 holes/cm². In an exemplary embodiment of the invention, the membrane pore diameter is in the range of between 0.1 or 0.05 microns to about 10 μm, optionally 1-3 μm, optionally about 1.7 microns.

In an exemplary embodiment of the invention, the member includes at least 10, 50, 100, 500 1000, 2000, 5000, 10,000, 50,000 or intermediate numbers of pores of the above sizes.

In an exemplary embodiment of the invention, the member includes a porous area of between 0.2 and 10 cm², for example, between 1 and 2 cm², between 1.5 and 5 cm² or intermediate areas.

Optionally, pore sizes are non-uniform in a patterned manner, for example, one or more lower diameter holes adjacent one or more higher diameter holes. Optionally, this is used to generate a slow jet surrounded by fast jest or vice versa. Optionally, there are at least 20% of pores of all the pores at each hole diameter of two or more pore diameters.

In an exemplary embodiment of the invention, system 100 (e.g., via controller 106) calculates an actual delivered dosage. Optionally, such delivery is presented in real time for a physician to follow. Optionally, the system integrates the rate of fluid delivery over time. Optionally or alternatively, the system takes into account initial leakage of fluid and/or fluid delivered at pressures too low to enter tissue. For example, the system may only take into account delivery when pressure is above a threshold. Optionally or alternatively, instead of measuring flow, the system measures pressure and estimates delivery according to know delivery rates at different pressures. Optionally or alternatively, the system presents estimated leakage into the blood stream. Optionally or alternatively, the system stops delivery and/or generates and alert when a desired amount of medicament is estimated to have been delivered or, possibly, a short time before such estimated delivery is completed.

In an exemplary embodiment of the invention, the balloon has a treatment length of between 8 and 80 mm, for example, between 10 and 30 mm or intermediate lengths.

In an exemplary embodiment of the invention, the catheter/shaft on which the balloon is mounted is of a length of between 5 and 200 cm, for example, between 100 and 150 cm. Optionally, the shaft diameter is less than 10 mm, 5 mm, less than 3 mm or intermediate sizes.

It should be noted that while the description herein focuses on using a balloon, which has the potential advantage that it can self expand to ensure contact with tissue, in some embodiments, medicament delivery is via an expanding element other than a balloon or via a non-expanding element. In one example, the delivery is via a membrane attached to a delivery system, which is urged against a tissue to be treated, but the membrane does not appreciably expand. In another example, delivery is via thin tubes, for example, with a diameter of 300-1000 microns and pores along their sides. Optionally, such tubes are urged against tissue to be treated using a balloon. Optionally or alternatively, systems with small pores as described in the art (possibly with a higher or lower surface density thereof) are used, albeit with pressures higher than suggested, for the express purpose of causing a desired jetting effect.

### Exemplary Medicament Flow Parameters

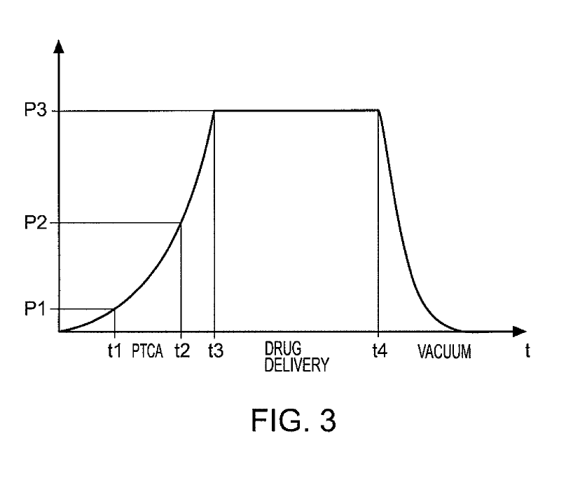

FIG. 3, described below, shows an exemplary pressure profile applied to a balloon. As the pressure is measured at a pressure source, it is noted that there can be a pressure drop of, for example, 10%, 30%, 50% or intermediate or greater amounts between the source and the balloon. Within such a profile there is a portion where medicament delivery is provided. In an exemplary embodiment of the invention, medicament is provided in a manner which will cause jets. Possibly, such jets penetrate tissue by forming an erosion hole. Different situations will reflect different desired erosion properties.

In an exemplary embodiment of the invention, the erosion hole has a depth between 0.001 mm and 0.2 mm, optionally between 0.01 mm and 0.05 mm. In an exemplary embodiment of the invention, the erosion hole is sized extends from an inner wall of a blood vessel into the endothelial layer, optionally until the area between the intima and media layers, and/or optionally extends into the media layer and/or beyond. Erosion hole depths may be controlled, for example, by setting fluid velocity, selecting pore size and/or selecting pressure and/or presence of eroding particles in the medicament.

In an exemplary embodiment of the invention, the average and/or maximal velocity of a single jet exceeds 0.1 m/s, optionally 0.5 m/s, optionally 5 m/s, or optionally exceeds 15 m/s, or is intermediate in velocity. An exemplary low flow rate per hole may be lower than 0.0001 cc/sec. As a function of balloon area, the flow rate may be, for example, lower than 0.1 cc/min/cm², and/or lower than 0.005 cc/sec/cm². In an exemplary embodiment of the invention, said flow rate is the maximal flow rate achieved during maximal injection pressure.

As noted above, in some embodiments of the invention, the intention is to achieve a desired minimal flow volume, without causing too much damage to tissue. For example, pressure and/or jet velocity may be reduced after it is estimated that there is a sufficient depth for erosion. In an exemplary embodiment of the invention, pressure and application duration are calculated according to a desired delivery amount and an allowed amount of tissue damage, for example, using tables or a function which inter-relates such parameters.

In some embodiments of the invention, the entire duration of the jetting forms a hole in the adjacent tissue. In other embodiments, a first part of the jetting forms a hole and a second part either only slowly increases the hole depth and/or size or does not affect the hole depth and/or diameter, but rather serves to provide additional material into the tissue. Optionally or alternatively, the jetting includes a series of one or more hole forming periods interspersed with material provisions. Optionally, the process is terminated with a hole forming act. Optionally, the acts being hole forming or material injection depends on the jet parameters which may be set, for example, by controlling the pressure (e.g., higher pressure for hole forming, for example, 300%, 200%, 100%, 50%, 30% or intermediate or higher percentages more pressure for hole forming).

In an exemplary embodiment of the invention, the duration of the hole-formation stage may be higher than 0.5 ms (milliseconds), optionally higher than 5 ms, optionally higher than 20 ms, optionally higher than 100 ms, optionally higher than 1 second or may be of any intermediate value. Alternatively, the hole forming stage is very long and may take over 5 seconds, optionally over 10 seconds. The duration of the medicament dispersion stage may be higher than 1 ms, optionally higher than 10 ms, optionally higher than 100 ms, optionally higher than 1 second, optionally higher than 10 seconds, or may be of any intermediate value. In an exemplary embodiment of the invention, most of the medicament is dispensed out of the balloon in a period between 1 to 60 seconds, optionally 5 to 30 seconds, optionally about 15 or 20 seconds. Times smaller than and/or intermediate the times described herein may be used for some embodiments.

In an exemplary embodiment of the invention, an exemplary procedure total leakage prior to jetting is less than 40%, 30%, 20%, 1%, 0.5% or intermediate percentages of the amount of fluid exiting the balloon during jetting into tissue.

In an exemplary embodiment of the invention, at least 20%, at least 50%, at least 80%, at least 90% or intermediate percentages of provided medicament are ejected from the balloon during the jetting phase. Optionally, remaining medicament is sucked out of the catheter and/or washed out (e.g., ejected optionally as jets) using saline or other washing fluid.

Optionally, the maximal injection pressure is in the range of 10-100 atmospheres, optionally in the range of 15-50 atmospheres.

Optionally, the suggested exemplary balloon is capable of producing a continuous stream of fluid medication that with sufficient impact for active diffusion of said medication to said passage wall (e.g., to provide drug diffusion volume which is substantially higher than passive diffusion, resulting in better adhering to—and/or penetration and/or absorption of medicament into tissue).

In an exemplary embodiment of the invention, the medicament is filtered during manufacture and/or prior to use, for example, using filters with passing properties as described above. For example, for preventing particles smaller than 2 microns or smaller than 0.5 microns, for example about 0.2 or 0.45 microns.

### Exemplary Method of Treatment

FIG. 1A is a schematic flow diagram of an exemplary method 1000 for treating a narrowed segment 3100 of body passage 3000, using, for example, exemplary balloon catheter 2000. FIGS. 2A-2D graphically illustrate operation of balloon catheter 2000 in accordance with exemplary method 1000. Each of FIGS. 2A-2D is a lateral view of balloon catheter 2000, in different configuration, in a blood vessel 3000, or other intrabody lumen. FIG. 3 illustrates a schematic Pressure vs. Time graph illustrating an exemplary operational sequence of a balloon catheter 2000 according to exemplary method 1000.

In an exemplary embodiment of the invention, prior to treatment, a physician selects treatment parameters, for example, including one or more of medicament, desired amount, desired concentration, desired release profile, desired in-tissue concentration, desired penetration depth, maximal allowed penetration depth, vessel diameter, vessel length to be treated and/or angular sector to be treated. The physician can then determine, for example, which medicament and which balloon design to use. Optionally, a plurality of different balloons with different parameters are available. Optionally, the determination by the physician uses a table or a calculator into which desired results are input and possible device/medicament/pressure profiles are provided as an output.

FIG. 2A illustrates a positioning 1100 of balloon 2200 next to stenosis 3100 or other tissue to be treated. In this position, balloon 2200 is substantially collapsed and/or deflated in order to better its maneuverability within body passages until reaching the treated area. Balloon catheter 2000 may be advanced to the desired location by any means known to art, including or excluding the use of guide-wire 2300. If the tissue to be treated is asymmetric (e.g., stenosis on only one side of vessel), the balloon may be selected to be a balloon with asymmetric treatment/penetration profile and then oriented as needed.

FIG. 2B illustrates an initial inflation act 1200 of exemplary method 1000, in which balloon 2200 is expanded and/or inflated and takes the general form of the volume captured within narrowing 3100. Balloon 2200 expansion occurs when fluid (e.g., medicament) is pressurized using pressurizing means (not shown) and fills balloon 2200 interior, thus outwardly presses its inner surface according to the applied pressure. Often, a minimal pressure P1 (see also FIG. 3) is needed at 1200, at least in the case when a minimal resistance is applied from balloon surroundings, and/or by the balloon's own resistance to expansion. In an exemplary case, when a stent is delivered and disposed in narrowing 3100, a higher inflation pressure P1 is needed. Usually pressure P1 of about 6 atmospheres or less in needed for initially inflating a balloon inside a coronary artery while opening a stent, but higher or lower pressures may be needed as well. The duration of act 1200 (time between t0 and t1, e.g., t1−t0) is very short and can be, for example, a few seconds (e.g., 1-5 seconds).

FIG. 2C illustrates an optional angioplasty act 1300 of exemplary method 1000, in which balloon 2200 is now pressurized to pressure P2 (P2>P1), whereby narrowing 3100 is substantially opened to a satisfactory degree chosen by the physician, and optionally to the general diameter of the adjacent opened segments of body passage 3000. Optionally, pressures of 8 to 18 atmospheres used for angioplasty of a stenotic coronary artery, while lower pressures (e.g., 3-9 atmospheres) may be used for opening stenotic peripheral arteries. Higher or lower pressure may be applied for different body lumens according to the vessel mechanical properties, the procedural protocol and/or according to the physician's decision. Duration t2−t1 may also be relatively short, for example, 10-30 seconds or up to a minute or according to standard angioplasty and the physician's discretion. In some cases, the balloon is selected so that it does not expand enough to permanently mechanically change tissue properties, as in PTCA.

FIG. 2D illustrates a drug delivery 1400 act of exemplary method 1000, in which balloon 2200 is further pressurized during a period of time from t2 to t3 until a pressure P3 is reached, and medicament starts dispersed out of balloon 2200 through pores 2210 as a plurality of medicament jets 4000. Optionally, P3 overlaps with P2, so there is at least some jetting during PCTA. Exemplary jets 4000 are schematically illustrated in FIG. 2E, as a magnification of a portion of FIG. 2D. FIG. 2E is a magnified view of a segment of exemplary balloon 2200 which is in contact with narrowed segment 3100, showing plurality of pores 2210 and plurality of jets 4000. In an exemplary embodiment of the invention, P3 is between 10 and 80 atmospheres, optionally between 15 and 50 atmospheres. Optionally, duration t3−t2 is less than 3-5 seconds, optionally less than a second or less than 200 ms. In an exemplary embodiment of the invention, duration t4−t3 of drug delivery act 1400 is longer than 3 seconds, 8 seconds, 15 seconds, 30 seconds, 45 seconds or 60 seconds or shorter or intermediate durations. Optionally, the duration is longer (e.g., 1-10 minutes, for example, about 5 minutes or intermediate durations) if the pressure is reduced during delivery, for example, to provide a train of pressure pulses. Optionally, t4−t3 is determined (optionally predetermined) according to treatment type, location and severity of the lesion, specific balloon design and/or applied pressure P3.

In an exemplary embodiment of the invention, medicament jets 4000 travel into the stenosis tissue at narrowing 3100. Optionally, the penetration depth is maintained within the stenosis layer (e.g., lipids and/or fibrotic tissue), but alternatively it may be desired to apply injection pressures that will promote penetration into deeper layers of passage 3000 (e.g., the intima layer and/or the media layer of a blood vessel).

Optionally, some or all medicament jets 4000 do not penetrate into narrowing 3100 but rather medicament adheres to the vessel wall and coats it, and is subsequently absorbed in the tissue. Optionally, specific adhesive properties of the medicament are previously set in order to better its adhering properties and durability. Optionally, the balloon is kept in place for a short time, to assist in adhesion. Optionally, a second material is extruded form the balloon, optionally at a lower pressure to assist adhesion. Optionally or alternatively, a tissue adhesion enhancer, for example, tissue adhesive, is provided as part of the medicament or as a second provided medicament.

At 1500 (t4), pressure is reduced and jetting stops. The balloon may be collapsed, for example by vacuum and withdrawn. As noted above, pressure during time period (t3 . . . t4) can be varied, for example to alternate eroding and non-eroding jetting and/or to massage fluid into the tissue and/or otherwise manipulate the tissue. Optionally or alternatively, pressure alternation is used to allow tissue to rest between injections. For example, a series of 1-5 second injections may be spaced out over several minutes. Optionally, such alternation is used to allow intermittent blood flow past the treatment region. Optionally or alternatively, blood flow past is allowed by a bypass tube (not shown) which may or may not be part of the catheter system.

Optionally or alternatively, the provision of multiple medicaments is supported, for example, by emptying the catheter, optionally washing with low pressure saline and then refilling the catheter with a different medicament, optionally at a high pressure. Optionally or alternatively, the balloon is partially collapsed and repositioned to a new region to be treated which is then treated with a same or different medicament.

Optionally or alternatively, the exemplary method and apparatus described herein may be used for the treatment of in-stent restenosis, where only angioplasty and local drug administration is preferred. In an exemplary embodiment of the invention, the method described herein is useful for bent or torturous blood vessels and/or for vessel junctions where placing a stent can be difficult or impossible.

### Exemplary Medicament

As described earlier, the methods and apparatus described herein may be used for many different types of drugs and/or other materials and/or disorders. In one exemplary embodiment of the invention an anti-proliferative agent is used to treat a blood vessel wall in order to prevent or lower the possibility of restenosis. Optionally, the agent is Paclitaxel (Taxol), optionally provided as an active ingredient in a solution (for example, Medixel®, by Medison Pharma Ltd, Israel). Alternatively, Sirolimus (Rapamycin) or a Sirolimus derivative (such as Tacrolimus) is used.

In an exemplary embodiment of the invention, one of the ingredients for the drug is an injectable Paclitaxel solution, with 30 mg (milligram)/5 ml (milliliter) Paclitaxel active agent (for example, the commercially available as Medixel® of Medison Pharma Ltd, Israel). The drug solution is then diluted with saline and optionally with contrast medium (e.g., VISIPAQUE™ by Amersham Health Ireland, for example, iodixanol) in an exemplary volumetric ratio of 1:3:1 or 1:3.25:0.75 (Taxol:Saline:Contrast Medium). Optionally, after dilution, in each 1 cc of prepared drug, there will be 1-1.4 mg, optionally about 1.2 mg Paclitaxel. Optionally, said solution also includes approximately 10% Cremophor EL and/or approximately 10% Dehydrate Ethanol. Optionally, 10%-15% of the medicament is contrast material.

The medicament may contain any diluting material, such as saline. Optionally, the operator uses different dilutions for different applications and/or different balloon designs. For example, when using a balloon with larger hole diameters a more viscous medicament may be used (e.g., by increasing the volumetric percentage of a contrast media material), optionally calibrated so as to achieve similar hole erosion properties as with a more diluted medicament that is delivered through a balloon having smaller hole diameters. Optionally, the overall delivered quantity may change in correlation to the change of medicament dilution, in order to set a requested dose of the active ingredient. In some circumstances, a higher viscosity of delivered material may be used in order to achieve unique parameters in a specific tissue. Optionally, other material (e.g., not a contrast medium) that changes medicament solution viscosity may be used, for example, a sufficient. Optionally or alternatively, an effective amount of a material that improves tissue adhesion is provided.

Different saline:contrast-medium ratios may be prepared according to the requested viscosity of the drug. Generally, the contrast medium material is substantially more viscous than saline and can be used to control the viscosity of the resulting medicament. The physician optionally prepares the preferred volumetric ratio according to a table of ratios-vs.-viscosities provided to him with the drug ingredients and/or the balloon catheter kit. Optionally or alternatively, the physician (or a technician or other user) is provided with several packs of different pre-mixed drug ingredients having different ratios, which will be differentiated according to final mixture viscosity and/or other desired properties. Optionally, the use of contrast material allows the monitoring the progress of the procedure and/or allows to consider and/or monitor tissue migration and/or drug diffusion over time.

In an exemplary embodiment of the invention, it is noted that even in a solution, aggregates can form. Optionally, the sizes of the particles (e.g., of Paclitaxel) and/or aggregates delivered with the drug influences the minimal pores diameter chosen for the perforated balloon. Optionally, an effective injection of a Paclitaxel-based drug is met with minimal and/or average pores diameter of 0.5 μm or more, optionally 0.8 μm or more, optionally 1 μm or more, optionally 1.5 μm or more, optionally 2 μm or more, or higher or lower or intermediate diameters.

In another exemplary embodiment of the invention, one of the ingredients for the drug is an injectable Sirolimus (Rapamycin) solution, with Sirolimus concentration of 1-1.4 mg/ml. The drug bulk substance (raw material, as, for example, is commercially available from Chunghwa Chemical Synthesis & Biotech Co. Limited, Taiwan) is optionally dissolved with 100% Ethanol and 15% Tween 80 to form a stock solution, which is further diluted with Saline at a ratio of 1:49 (Saline:Stock solution). Rapamycin solution may be prepared using different solvents, for example, as described in US Patent Application 2005/0222191.

In tests performed by the inventors it was determined that a balloon having pores with diameter of 1.7-2 μm, enables delivery of Taxol solution or saline as jets via its pores.

In accordance with exemplary embodiments of the present invention, the administered material may consist, for instance, of compounds or drugs selected for one or more of reducing cell division activity (e.g., Paclitaxel, Rapamycin, and/or their derivates), vasomotor action (calcium antagonists) and inflammatory response (steroids) as well as anticoagulants. Calcium antagonists may include materials such as diltiazem HCl, nifedipine and verapamil HCl, steroids such as dexamethasone and specific nonsteroidal anti-inflammatory agents. Anticoagulants may include materials such as heparin, hirudin, dipyridamole, papaverine HCl, ethaverine HCl and prostacyclin inhibitors. It is also contemplated that agents (e.g., antisense, growth inhibitor, or gene therapy) inhibiting smooth muscle proliferation, which is, apparently, a primary factor in restenosis, or agents tending to reduce collagen response to injury could also be used. Fibroblast proliferation inhibiting agents may also be included as well as collagen response reduction agents. It is still further contemplated that compounds that reduce platelet aggregation may also be beneficial to administer. Also, antitumor or other antimitogenic agents can be used for prevention of restenosis. Optionally, a combination of more than one drug/material may be administered.

In case of tumors treatment the medicament may include, for example, a drug such as mechlorethamine, cyclophosphamide, chlorambucil (leukeran), melphalan (alkeran), busulfan (myleran), dacarbazine (DTIC), cisplatin (Platinol), methotrexate, 6-mercaptopurine 6-MP, thioguanin 6-TG, 5-fluorouacil (5-FU), vinblastine (velban), dactinomycin, doxorubicin, daunorubicin, mitomycin (mutamycin), diethylstilbestrol, and retinoic acid and analogues. Some embodiments of the present invention are suited to delivery of sensitizer and immunomodulator drugs. Optionally, more than one drug/material listed above and/or other material may be administered.

In an exemplary embodiment of the invention, medicament may include gene therapy. In another exemplary embodiment, medicament may include angiogenesis factors.

In an exemplary embodiment of the invention, the medicament is a structure affecting medicament, for example a material which stiffens, softens and/or makes tissue more or less elastic. Optionally, the medicament is a tissue adhesive.

In an exemplary embodiment of the invention, the medicament is an ablating material, for example, which kills tissue, for example, a high concentration of ethanol.

It should be noted that in some embodiments of the invention, tissue modifying effects are achieved using injection of saline.

In an exemplary embodiment of the invention, the medicament is provided encapsulated and/or attached and/or adsorbed with particles, to provide for its slow release following administration. Optionally, the microencapsulation particles (for example, PGA, PLA, PGLA, PCL), are smaller than the size of balloon pores. In an exemplary embodiment of the invention, Paclitaxel is delivered encapsulated within particles having a diameter smaller than 1 μm. Optionally, particle diameter is in the range of 50-300 nm. Optionally, said particles aggregate to form larger particles, having a diameter smaller than 3 μm, optionally smaller than 1 μm. Optionally, the particles are smaller than 70%, 50%, 30%, 20%, 10% or smaller or intermediate percentages of the pore diameter. Optionally or alternatively, some pores are smaller than particles and do not pass particles, only jets. Alternatively, only part of the drug, for example Paclitaxel, is provided encapsulated and is slow released, while the rest of the drug is free and immediately penetrates into—and/or adheres to the tissue following administration. Optionally, a total volume of 0.01-0.3 cc is injected throughout the complete procedure, optionally 0.03-0.20 cc, for example, for a treatment area of 1-2 cm², for example, about 1.8 cm². Optionally, the resulting concentration by volume in the target tissue is between 0.1% and 30%, for example, between 1% and 10% or intermediate percentages. Optionally, the volume which penetrates into the body passage wall is at least 1%, optionally at least 5%, optionally at least 20%, optionally at least 50% of the total injected volume.

In an exemplary embodiment of the invention, the drug used is Rapamycin (Sirolimus), encapsulated within biodegradable particles, which optionally have a diameter smaller than, for example, 400 nm, 200 nm, 100 nm and/or intermediate diameters and/or are optionally made of a polymer, such as PLLA (poly, L lactic acid) or PLGA (poly lactic glycolic acid). Optionally, the particles are prepared using a solvent evaporation technique.

In an exemplary embodiment of the invention, the drug is released from the particles in a slow release manner (for example, over 3-4 weeks for restenosis or over other time periods for other applications, which can be, for example, 1-4 days, 5 weeks, 3 months or shorter, intermediate or longer periods. Optionally, the initial delivery is delayed, for example, for 2-3 days, to allow healing of the jet-caused wounds and/or stents or PTCA caused damage.

In an exemplary embodiment of the invention, the encapsulated particles are supplied as a powder, which is mixed with and suspended in sterile distilled water/glucose solution immediately prior to use. Optionally, drug concentration (in the mixture) is about 100 μg/ml or about 1 mg/ml. Optionally, such particles with drug are manufactured by Southwest Research Institute, San Antonio, Tex., USA.

A potential advantage of using particles rather than a solution of a drug is that the drug in the solution is more likely to react and/or aggregate than particles within which, drug is encapsulated and not available for reaction.

Optionally, the delivered material or medicament has lipophilic and/or tissue adhering properties (optionally selectable by changing the formulation) so at least part of the injected volume can coat the inner wall surface of the body passage. Optionally, at least part of the injected material is attached to—and/or penetrates the body passage wall for at least 5 seconds, optionally at least 30 seconds, optionally at least 1 minute, optionally at least 1 hour, optionally at least 1 day, optionally at least 10 days. Optionally, said material is degraded over time and/or is biodegradable, while preserving residual quantity for few hours or a few days, or in any lesser or higher or intermediate values.

In an embodiment, the medicament is coupled/bound to a carrier, suitable for the delivery of the medicament to/into the target site (e.g., endothelial cells of blood vessel wall). In an exemplary embodiment of the invention, a protein, such as Albumin, which is a natural carrier of lipophilic molecules, serves as a delivery vehicle for the drug (as done by Abraxis Bioscience, for example, using ABRAXANE™ or Nab™ technology). Optionally, an insoluble or poorly soluble drug may be combined with such a protein, to form a nanoparticle and to facilitate drug solubility and delivery. Optionally, no toxic agents that are normally being used as solvents are required for the process.

In an exemplary embodiment of the invention, the medicaments and/or delivery systems are packaged with instructions and/or labeled for specific applications and/or usage protocols. Optionally, a label on the medicament and/or a label on the delivery catheter are read by the delivery system and this information (e.g., medicament properties and/or balloon properties) is used to configure, optionally automatically, the pressure profile used for delivery.

### Exemplary Manufacturing Method

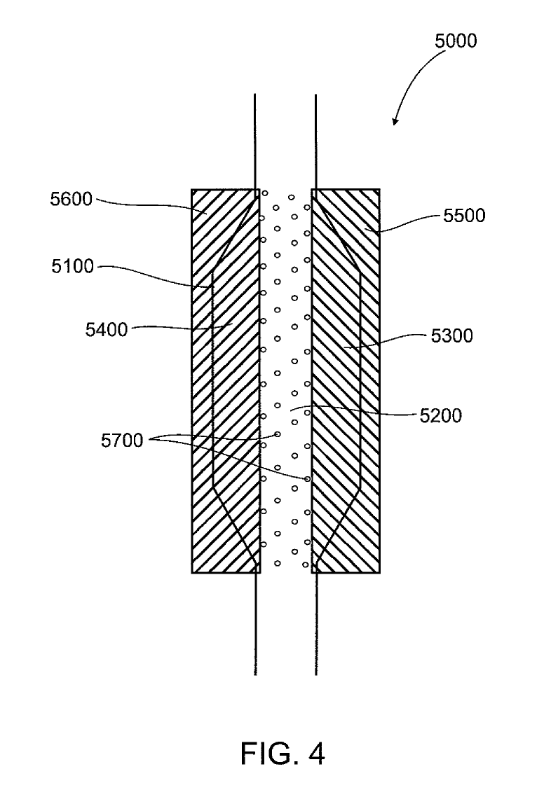

FIG. 4 illustrates a method to produce perforated balloon 5000 in which most of balloon microholes are approximately perpendicular to balloon wall 5100. Balloon perforation is performed, for example, using a track-etch process, in which a thin polymer film (e.g., balloon membrane) is bombarded by charged particles (protons, ions, electrons or other radiation), and then subsequently subjected to a controlled etching, during which the tracks left by the particles are preferentially etched, optionally to form uniform, cylindrical pores of predetermined size.

In FIG. 4, balloon 5000 is rotated, for example by connecting one or both ends of balloon to a dedicated apparatus (not shown in FIG. 4), that turns the balloon in a controlled, optionally predefined, rate, as required. Balloon portion 5200 that is positioned substantially perpendicular to the perforation source (e.g., a nuclear reactor/cyclotron; not shown in FIG. 4) remains exposed, while other portions (5300, 5400) of balloon surface, which do not face the perforation source are shielded from the perforation source (for example, by metal such as a lead shield 5500 5600). The perforation source is activated, and the unshielded portion 5200 of balloon surface is bombarded, to create microholes 5700 that are angled at approximately 90 degrees to balloon surface at the perforation point. Optionally, a perforated mask is sued to set the location, pattern, size and/or other properties of the pores. Then, balloon 5000 is rotated, to expose a new surface that was previously shielded and is currently positioned perpendicular to the perforation source, and to shield other surface of balloon that is no longer perpendicular to the perforation source and has already been perforated. The perforation source is re-activated and the newly-exposed area is bombarded to perforate it at approximately 90 degrees. The balloon 5000 is rotated again, and the procedure is repeated a few times, until all of—, or a desired part of the balloon is bombarded while being positioned approximately perpendicular to the perforation source. Optionally or alternatively, axial masking may be used as well. In an exemplary embodiment of the invention, the beam of the radiation source comprises a plurality of beams (e.g., formed by masking a wide aperture beam with a mask and/or by relative movement of a narrow beam and the balloon), optionally parallel, but alternatively non-parallel, for example, diverging are converging in one or two dimensions.

### Exemplary Urethral and Other Treatment

Any number of illnesses may be treated using exemplary method and apparatus in accordance with the present invention, including treatments of narrowed or non-narrowed body lumens. These may include: treatment for the prevention of restenosis or any other possible narrowing of a lumen, delivery of drugs to treat cancer (that may be evolving in, for example, biliary duct, trachea, eosophagus), delivery of drugs to prevent hyperplasia (e.g., in the case of BPH treatments; such drugs may include anti-androgen to prevent prostate hyperplasia, Botox for tonus relaxation, and cytotoxic drugs for local treatment of hyperplasia and/or cancer), delivering anesthetics to a target area before a treatment (e.g., as needed in some invasive treatments in the urethra).

In an exemplary embodiment of the invention, the microporous balloon of the invention is used to open a urethral stricture as well as drug injection into the urethra wall at the stricture site. Optionally, balloon is introduced transurethrally. Optionally, balloon is introduced into the urethra via a working channel of an endoscope. Optionally or alternatively, the procedure is performed under fluoroscopy, and balloon visualization is enabled by the addition of a contrast medium to the drug solution.

Various embodiments and aspects of the present invention as delineated hereinabove and as claimed in the claims section below may find experimental support in the following examples.

### EXAMPLES

Reference is now made to the following examples, which together with the above descriptions, illustrate some embodiments of the invention in a non limiting fashion.

### Example 1

### Balloon with 0.8 μm Diameter Holes

A first exemplary balloon includes pores with a 0.8 μm diameter and a density of 5,000 pores/cm². The balloon is based on a PET membrane perforated using track-etching technique. The balloon diameter in expanded form is approximately 3 mm and has a wall thickness of possibly 20 microns.

Two in-vitro tests were performed with this balloon type on tissue (domestic pig coronary arteries), using different injection parameters (as detailed below). All injections used same drug composition of 1:3:1 (taxol:saline:CM) ratio with 1.2 mg/ml Paclitaxel concentration. The tissues were then deep frozen and underwent HPLC examination for evaluation of the penetrated Paclitaxel quantities of each injection.

Test No. 1 included a continuous drug delivery with pressure (P3) of 10 atmospheres during 60 seconds (t4−t3); and Test No. 2 had a P3 of 18 atmospheres and t4−t3 of 15 seconds. The HPLC results showed that in Test No. 1 the total amount of Paclitaxel found in the tissue was 392.8 ng (nanogram) (about 0.49 weight % of the total injected medicament), and in Test No. 2 a quantity equal to 2,520.4 ng was traced in the tissue (about 5.6 weight % of the total injected medicament). For both tests, no substantial damage to tissue was revealed in histological examination. These test results suggest that there is a correlation between the magnitude of the drug delivery pressure (P3) and the effectiveness of the treatment (e.g., the amount of drug adheres and/or penetrates into tissue), at least with respect to the specific balloon perforation design (e.g., 0.8 micron holes and 500 holes/cm²). Drug delivery at a higher pressure of 18 atmospheres resulted in a significantly higher (non-linear) drug penetration level into tissue. This can be useful, for example, in that if a higher rate of delivery is desired and/or if delivery to a nearby tissue is desired, pressure can be increased (e.g., temporarily). Optionally, depth of penetration is controlled using a table linking pressure to penetration and selection of a pressure according to a desired penetration depth.

### Example 2

### Comparison Between Exemplary Microporous and Macroporous Balloons

The following is a comparison between a microporous balloon in accordance with an exemplary embodiment of the present invention and an exemplary macroporous balloons. The microporous balloon includes 1.5 μm diameter pores with density of 1,000 pores/cm² (a total of 1,880 pores). The chosen macroporous balloon includes 88 pores of 20 μm (microns) in diameter.

The use of said macroporous balloon with the particular pressure source used did not enable elevation of the pressure to 10 atmospheres, due to the relatively large diameter pores of said balloon, compared to the catheter lumen diameter.

In order to overcome the problem of pressure elevation, a second macroporous balloon having a double balloon design (with the inner balloon serving as a valve to the outer balloon) was tested (balloons with 88-160 pores, each having a diameter of 8-20 μm and pore density of 62-113 pores/cm²). Using this balloon, a volume of 0.025cc saline solution was injected in a pulse of 0.02 seconds. The injection pressure (pulse) was approximately 22-25 atmospheres. The derived overall flow rate was 1.25 cc/sec and the derived jet speed was 45.2 m/sec.

Several in-vitro tests performed with the macroporous balloons using similar parameters (injected into the walls of coronary arteries of domestic pigs) showed more difficulty with controlling penetration depths and distribution of the drug.

When injecting a quantity of 0.15 cc drug solution with the exemplary microporous balloon (for example when P3=18 atmospheres and t4−t3=60 seconds), the overall flow rate is 0.0025 cc/sec (or 0.00133 cc/(sec×cm²)), and the jet velocity is approximately 0.75 m/sec. Histological examination following said injection did not detect damage to tissue, while HPLC results indicated drug presence in tissue.

### Example 3

### Balloon with 1.7 μm Diameter Holes

Additional tests were performed with another exemplary microporous balloon, having 1.7 μm diameter pore with pore density of 550 pores/cm² (a total of 1,036 pores). When injecting a quantity of 0.185 cc Taxol solution (with concentration of 1.2 mg/ml and 15% contrast medium as described above) using this exemplary balloon, under P3=18 atmospheres and t4−t3=20 seconds, the overall flow rate is 0.00925 cc/sec (and a flow rate of 0.00000892 cc/sec for a hole), and the jet velocity is approximately 3.93 m/sec.

### Additional Exemplary Tests

The following table (Table 1) presents flow rate and velocity results obtained while injecting various amounts of Taxol solution (in concentration of 1.2 mg/ml), using microporous balloons, under various pressures and durations. The tests were performed with balloons having 20 mm length and 3 mm diameter, with various pore size and pore density, as specified in the table below. The flow rate and pore flow rate are calculated based on the other columns.

TABLE 1

| | | De- | | | | |
|---|---|---|---|---|---|---|
| | Pore | livery | De- | De- | | |
| Pore | Density | Pres- | livery | livery | | |
| Size | [Pore/ | sure | Time | Volume | Total Flow | Pore Flow Rate |
| [μm] | cm²] | [Atm] | [sec] | [cc] | Rate [cc/sec] | [cc/sec] |
| 0.8 | 5000 | 15 | 60 | 0.055 | 0.000916667 | 0.0000000931 |
| 2.5 | 400 | 16 | 15 | 0.253 | 0.016866667 | 0.0000223815 |
| 2 | 550 | 16 | 15 | 0.12 | 0.008 | 0.00000772052 |
| 2 | 550 | 12 | 15 | 0.11 | 0.00733333 | 0.00000707714 |
| 2 | 550 | 20 | 15 | 0.27 | 0.018 | 0.0000173712 |
| 2 | 550 | 12 | 30 | 0.19 | 0.00633333 | 0.00000611208 |
| 2 | 550 | 20 | 30 | 0.6 | 0.02 | 0.0000193013 |
| 1.9 | 550 | 12 | 15 | 0.0726 | 0.00484 | 0.00000467091 |
| 1.9 | 550 | 20 | 15 | 0.2 | 0.01333333 | 0.0000128675 |
| 1.7 | 550 | 8 | 20 | 0.0037 | 0.000185 | 0.00000017853 |
| 1.7 | 550 | 18 | 20 | 0.185 | 0.00925 | 0.00000892685 |
| 1.7 | 550 | 20 | 20 | 0.216 | 0.0108 | 0.0000104227 |

The following table (Table 2) presents test results of the amount of delivered drug (Taxol with 15% contrast medium) from a 20 mm long, 3 mm diameter balloon, with 1.7 μm diameter pore and pore density of 550 pore/cm². The drug amount is presented as function of pressure and time. In the table, only the pressure, time and amount were measured and the other columns were calculated/estimated.

TABLE 2
Delivered Drug Amount [μg]

| | Pressure | | | | |
|---|---|---|---|---|---|
| Time | [Atm] | | | | |
| [sec] | 8 | 12 | 16 | 18 | 20 |
| 1 | 0.2 | 1 | 1 | 12 | 14 |
| 2 | 0.5 | 2 | 2 | 23 | 27 |
| 3 | 1 | 3 | 3 | 35 | 41 |
| 4 | 1 | 4 | 5 | 47 | 55 |
| 5 | 1 | 5 | 6 | 59 | 68 |
| 6 | 1 | 6 | 7 | 70 | 82 |
| 7 | 2 | 7 | 8 | 82 | 96 |
| 8 | 2 | 8 | 9 | 94 | 109 |
| 9 | 2 | 9 | 10 | 105 | 123 |
| 10 | 2 | 10 | 11 | 117 | 137 |
| 11 | 3 | 11 | 12 | 129 | 151 |
| 12 | 3 | 12 | 14 | 141 | 164 |
| 13 | 3 | 13 | 15 | 152 | 178 |
| 14 | 3 | 14 | 16 | 164 | 192 |
| 15 | 4 | 15 | 17 | 176 | 205 |
| 16 | 4 | 16 | 18 | 187 | 219 |
| 17 | 4 | 17 | 19 | 199 | 233 |
| 18 | 4 | 18 | 20 | 211 | 246 |
| 19 | 4 | 19 | 22 | 222 | 260 |
| 20 | 5 | 20 | 23 | 234 | 274 |
| 21 | 5 | 21 | 24 | 246 | 287 |
| 22 | 5 | 22 | 25 | 258 | 301 |
| 23 | 5 | 23 | 26 | 269 | 315 |
| 24 | 6 | 24 | 27 | 281 | 328 |
| 25 | 6 | 25 | 28 | 293 | 342 |
| 26 | 6 | 26 | 29 | 304 | 356 |
| 27 | 6 | 28 | 31 | 316 | 370 |
| 28 | 7 | 29 | 32 | 328 | 383 |
| 29 | 7 | 30 | 33 | 340 | 397 |
| 30 | 7 | 31 | 34 | 351 | 411 |

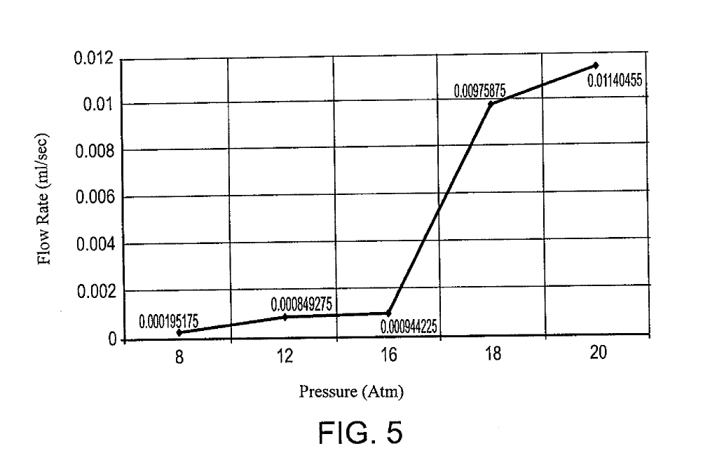

FIG. 5 shows the dependence of flow on the pressure. As can be seen, there is a sudden change in flow rate between 16 atmospheres and 18 atmospheres pressure, for a 3×20 mm balloon. The following table summarizes the data shown in the graph. It is expected that the change in rate can be controlled, for example, by controlling the fluid viscosity and/or pore size.

| | Weight after | | | |
|---|---|---|---|---|
| pressure | 20 seconds | volume after 20 | flow rate | drug rate (est) |
| (atm) | (gr) | seconds (ml) | (est) ml/sec | mg/sec |
| 8 | 0.0037 | 0.0039035 | 0.000195175 | 0.00023421 |
| 12 | 0.0161 | 0.0169855 | 0.000849275 | 0.00101913 |
| 16 | 0.0179 | 0.0188845 | 0.000944225 | 0.00113307 |
| 18 | 0.185 | 0.195175 | 0.00975875 | 0.0117105 |
| 20 | 0.2162 | 0.228091 | 0.01140455 | 0.01368546 |

In additional series of tests, the inventors evaluated the potential damage to the treated tissue following the use of the discussed microporous balloon system.

As an initial detremination, in vitro tests were performed using a 20 mm long, 3 mm diameter balloon, with pore diameter of 1.7 microns and pore density of 550 pore/cm². The balloon, mounted on a PTCA catheter, was introduced into pig coronary arteries, and the pressure was elevated to about 10-12 Atm (with over-dilatation of 10%), to simulate an angioplasty procedure. Then, the pressure was elevated to 18 Atm for about 30 seconds, and a volume of about 0.15 cc of saline solution with ink was injected. The ink dye indicated the material penetrates into the blood vessel wall, into the intima and further into part of the media layer.

Following said in vitro tests, an in vivo procedure was performed in pigs, under the same protocol (i.e., initial pressure of about 10-12 Atm with over-dilatation of 10%, and then elevation of the pressure to 18 Atm for about 30 seconds, with the exception that ink was not added to the saline solution). The same balloon was used. After a week, the animals were sacrificed, and the treated tissue was histological assessed in a certified laboratory. The histological examination did not detect any significant damage, inflammation signs or injury to the vessel wall. This result suggests that it is possible to inject material into a vessel wall without causing damage that would be problematic or encourage restenosis.

It is expected that during the life of a patent maturing from this application many relevant medicaments for affecting tissue structurally and/or functionally will be developed and the scope of the term medicament is intended to include all such new technologies a priori.

As used herein the term “about” refers to ±10%. Such limitation is optionally applied to any numerical value described herein.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A medicament delivery system comprising a delivery unit, comprising:
a chamber having at least one wall, wherein said wall defines at least 10 pores with a pore diameter between 1 to 5 μm and a surface pore density of 300-10,000 pores/cm².

2. A system according to claim 1, wherein said chamber comprises a balloon.

3. A system according to claim 2, wherein said balloon is mounted on a catheter.

4. A system according to claim 1, comprising a pressure source fluidicly connected to said chamber.

5. A system according to claim 4, comprising a filter between said pressure source and said wall, said filter configured to pass particles smaller than 2 microns.

6. A system according to claim 4, wherein said pressure source is configured to provide both a first pressure suitable for expanding a passageway and a delivery pressure of at least 4 atmospheres greater than said passageway expanding pressure for delivering a medicament.

7. A system according to claim 6, wherein said delivery pressure is sufficient to cause jetting of a medicament contained in said chamber, with properties suitable for penetrating into a blood vessel wall.

8. A system according to claim 7, comprising a controller which controls one or both of said delivery pressure and a duration of said delivery to control an amount of delivered medicament.

9. A system according to claim 8, wherein said duration is between 5 and 60 seconds.

10. A system according to claim 7, wherein said jetting is suitable for penetrating past said blood vessel wall.

11. A system according to claim 1, containing an anti-proliferative agent suitable for the prevention of restenosis.

12. A system according to claim 1, sized for insertion into one or more of a urethra, a trachea, a ureter, an eosophagus esophagus, an ileum, a biliary duct, a fallopian tube, a tear duct and a nasal cavity.

13. A system according to claim 1, wherein said pore size is between 1.4 and 2 microns.

14. A system according to claim 1, wherein said pore density is between 300 and 600 pores/cm² for an area of at least 0.5 cm².

15. A system according to claim 1, wherein said pore density is about 550 pores/cm² and said pore size is about 1.7 microns in diameter.

16. A system according to claim 6, wherein said delivery pressure is at least 15 atmospheres.

17. A system according to claim 1, wherein said chamber is non-compliant.

18. A system according to claim 1, wherein said chamber is filled with a fluid including a plurality of particles configured to slowly release a medicament.

19. A system according to claim 1, packaged as a kit with medicament suitable for treating tissue.

20. A system according to claim 1, wherein at least 70% of said pores are oriented within 20 degrees of a perpendicular to said membrane.

21. A system according to claim 1, including a radio-opaque material in an amount suitable for fluoroscopic imaging, adjacent or in said chamber.

22. A system according to claim 1, comprising a unit which displays an estimate of an actually delivered amount of medicament.

23. A system according to claim 1, wherein said wall defines at least 100 pores and wherein at least 90% of pores in said wall are smaller than 5 microns in diameter.

24. A system according to claim 1, wherein:
said chamber is a balloon having at least 50 pores formed therein, said pores having a diameter of less than 5 microns,
said balloon being filled with a medicament under a pressure suitable for causing jetting of said medicament through said pores into tissue to a depth of at least 0.1 mm.

25. A system according to claim 24, wherein said pores have a diameter of less than 2 microns.

26. A system according to claim 24, wherein said delivery pressure is at least 8 atmospheres.

27. A system according to any claim 24, wherein said balloon is suitable for PTCA.

28. A system according to claim 24, wherein said medicament is suitable for preventing restenosis when injected into vascular tissue.

29. A system according to claim 24, wherein said medicament includes radio-opaque contrast medium.

30. A method according to claim 50, for treating a segment of a body lumen, further comprising:
(a)
(b) inflating said member to a first pressure sufficient to widen a narrowing in said segment, but not sufficient to cause significant leakage out of said pores,
wherein said inflation to said first pressure is performed before inflation to said delivery pressure; and
(c) further inflation of said member to said delivery pressure does not significantly increase a diameter of said member but is sufficient to cause jetting of a medicament out of said pores and into said tissue to a depth of at least 50 microns.

31. A method according to claim 50, wherein said surface has a pore density in the range of 300-10,000 pores/cm² for an area of at least 0.5 cm² and pore diameters in the range of 1-5 μm.

32. A method according to claim 50, comprising applying said jetting at a velocity and for a time suitable to form medicament reservoirs in said tissue.

33. A method according to claim 30, wherein said medicament is configured to adhere to said narrowing.

34. A method according to claim 30, wherein said inflation to said further pressure is performed immediately after inflation to said first pressure without interruption.

35. A method according to claim 30, wherein the first inflation pressure is between 5 and 12 atmospheres.

36. A method according to claim 50, wherein the delivery pressure is between 10 and 50 atmospheres.

37. A method according to claim 30 wherein said delivery pressure is provided for between 5 and 60 seconds.

38. A method according to claim 50, wherein the medicament includes an anti-proliferative agent in an amount suitable for the prevention of restenosis.

39. A method according to claim 30, wherein an amount of leaking medicament during inflation to said first pressure is less than 20% of an amount exiting said member by said further inflating.

40. A method according to claim 30, comprising deploying a stent in said narrowing using said inflatable member.

41. A method according to claim 30, for the treatment or prevention of in-stent restenosis.

42. A method according to claim 50, for the treatment of a blood vessel following arterectomy.

43. A method according to claim 30, comprising displaying at least an estimation of jetted medicament to a user during said further inflation, in real time.

44. A method according to claim 30, comprising imaging said narrowing during said further inflation, using a radio-opaque material adjacent or coupled to said inflatable member.

45. A method according to claim 30, wherein said further inflating does not cause tissue damage significant enough to cause restenosis.

46. A method according to claim 30, wherein said further inflating comprises injecting at least 0.025 ml/cm² medicament into tissue adjacent said narrowing.

47. A method according to claim 30, wherein said further inflating comprises injecting at least 0.07 ml/cm² medicament into tissue adjacent said narrowing.

48. A method according to claim 50, wherein said pores have a diameter less than 5 microns.

49. A method according to claim 50, wherein said medicament is slowly released into tissue, over a period of at least 5 days.

50. A method for treating a body portion, comprising:
(a) locating a chamber having a plurality of pores smaller than 5 microns formed in a surface thereof, adjacent said portion;
(b) filling said chamber with a quantity of medicament;
(c) pressurizing said chamber to a delivery pressure which is sufficient to cause jetting of said medicament out of said pores and into adjacent tissue to a depth of at least 50 microns with no significant tissue damage.

51. A method for producing a microporous balloon having at least 50% of pores at a desired orientation, comprising:
providing at least one perforation source;
shielding at least a part of a membrane surface that is not oriented within a desired angular range of said at least one perforation source;
activating said at least one perforation source;
exposing a different portion of said membrane to said at least one perforation source; and
repeating said activating and said exposing until a desired pattern of intended perforations of perforation sites is formed in said membrane.

52. A method according to claim 51, wherein said membrane is a balloon and wherein said balloon is exposed by rotating said balloon.

53. A method according to claim 51, wherein said desired orientation and said at least one perforation source are selected so that said intended perforation sites are substantially perpendicular to a surface of said membrane.

54. A method according to claim 51, wherein said source comprises a radiation source suitable for weakening a molecular structure of said membrane and comprising chemical etching of said membrane to convert intended perforation sites formed by said weakening into actual perforations.

55. A method according to claim 51, wherein said perforations have a diameter of less than 5 microns and said membrane has a thickness of at least 10 microns.

56. A method according to claim 50, comprising:
filtering said medicament; and
extruding said filtered fluid through pores in a microporous balloon.

57. A method according to claim 56, wherein extruding comprises extruding as jets which penetrate into tissue and form fluid reservoirs therein.

58. A method according to claim 56, wherein filtering comprises filtering after said medicament passes a perimeter of the human body.

59. A drug delivery system according to claim 1, wherein:
a delivery head including said chamber includes at least 50 pores each having a diameter of less than 5 microns formed therein;
a pressure source capable of reaching over 4 atmospheres and fluidicly connected to said chamber; and
a filter fluidicly located between said pressure source and said delivery head and configured to pass only particles smaller than 2 microns.

60. A system according to claim 5, wherein said filter is adjacent or in said delivery head.

61. A system according to claim 59, wherein said head comprises an intravascular catheter.

62. A pressurized medicament delivery perforated balloon that is filled with medicament and a fluidic contrast medium, in relative amounts that produces both radiographic visibility of said balloon when within a body and a viscosity suitable for jetting into tissue via pores of said balloon at a delivery pressure under which said balloon is pressurized.

63. A balloon according to claim 62, wherein said mixture includes an anti-proliferative agent used for the prevention of restenosis.

64. A method according to claim 30, further comprising, before a):
d) selecting a desired treatment including a desired medicament an a delivery unit including a delivery chamber;
e) determining a desired viscosity for said medicament and
f) formulating a mixture having said viscosity by mixing at least a medicament and a contrast material.

65. A method according to claim 64, wherein said formulating comprises also adding a diluting material.

66. A method according to claim 64, wherein said chamber is in the form of a perforated balloon, and said formulating and said determining are according to one or more of the balloon's average pores diameter, balloon number of pores, medicament delivery pressure and medicament delivery duration.
